(19) United States
(12) Patent Application Publication
(10) Pub. No.: US 2011/0046102 A1
(43) Pub. Date: Feb. 24, 2011

(54) AZABICYCLIC COMPOUNDS, PREPARATION THEREOF AND USE OF SAME AS DRUGS, ESPECIALLY BETA-LACTAMASE INHIBITORS

(75) Inventors: Benoit Ledoussal, Bouleurs (FR); Marie-Edith Gourdel, Savigny Le Temple (FR)

Correspondence Address: David Chen, IP Attorneys Group, LLC, 57 Plains Road, Suite 3A, Milford, CT, 06461, US

(73) Assignee: Novexel, Romainville (FR)

(21) Appl. No.: 12/990,312

(22) PCT Filed: Apr. 24, 2009
(86) PCT No.: PCT/IB2009/005391

§ 371 Date: Oct. 29, 2010

(30) Foreign Application Priority Data
Apr. 29, 2008 (FR) 08/02395

(51) Int. Cl. C07D 471/08; A61K 31/439; A61K 31/546; A61P 31/04

(52) U.S. Cl. 514/203; 546/121; 514/300

(57) ABSTRACT

The invention concerns the compounds meeting formula (I:

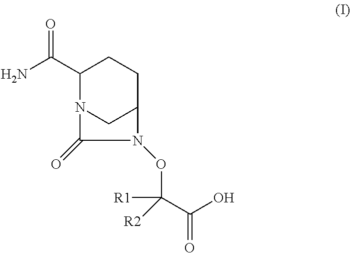

wherein one of R₁ and R₂ is a hydrogen and the other a fluorine or both represent a fluorine, in free form, in the form of zwitterions and of salts with pharmaceutically acceptable mineral or organic bases, the preparation thereof and their use as medicinal products inhibiting the action of β-lactamases by pathogenic bacteria.

The invention concerns novel heterocyclic compounds, the preparation thereof and their use as medicinal products, notably as inhibitors of β-lactamases action by pathogenic bacteria.

Application WO02/10172 describes azabicyclic compounds and their salts with a base or an acid, meeting formula A:

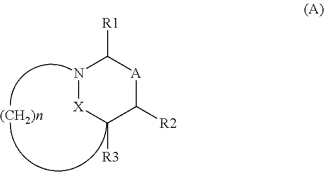

wherein:

R₁, R₂, R₃, A, X and n′ are defined in the application, and notably compounds in which X is a divalent —C(O)—B— group attached to the nitrogen atom by the carbon atom, and B is a divalent —NR₈— group attached to the carbonyl by the nitrogen atom, R₈ being chosen from the group comprising hydrogen OH, R, OR, Y, OY, Y, OY₁, Y₂, OY₂, Y₃, OCH₂CH₂SOₘR, OSiRₐR_{b}R_{c} and SiRaRbRc, R, Y, Y₁, Y₂, Y₃, m, Ra, Rb and Rc being defined in the application.

R₈ may therefore notably represent a OSO₃H or O—CH₂—COOH radical.

The compounds described in application WO02/10172 have anti-bacterial properties.

Application WO03/063864 describes the use of compounds (A) as inhibitors of β-lactamases and their combination with β-lactamines.

Applications WO02/100860 and WO04/052891 describe azatricylic compounds meeting formula B:

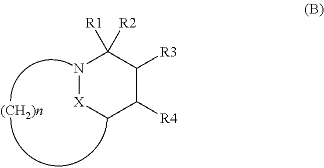

differing from compounds (A) notably through R₃ and R₄ which together form a phenyl or heterocycle of aromatic type, optionally substituted.

The subject of the present invention concerns compounds meeting formula (I):

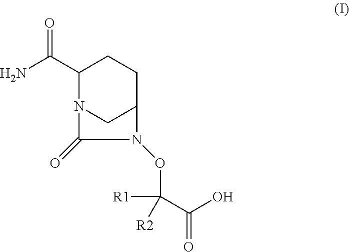

wherein one of R₁ and R₂ is a hydrogen and the other a fluorine, or both are a fluorine in free form, in the form of zwitterions and of salts with pharmaceutically acceptable mineral or organic bases.

Among the base salts of the formula (I) products mention may be made inter alia of those formed with mineral bases such as sodium, potassium, lithium, calcium, magnesium or ammonium hydroxide, or with organic bases such as methylamine, propylamine, trimethylamine, diethylamine, triethylamine, N,N-dimethylethanolamine, tris (hydroxymethyl)amino methane, ethanolamine, pyridine, picoline, dicyclohexylamine, morpholine, benzylamine, procaine, lysine, arginine, histidine, N-methylglucamine, or phosphonium salts such as alkyl-phosphoniums, aryl-phosphoniums, alkyl-aryl-phosphoniums, alkenyl-aryl-phosphoniums or quaternary ammonium salts such as the salt of tetra-n-butyl-ammonium.

The asymmetric carbon atoms contained in the formula (I) compounds may each independently have R, S or RS configuration, and a further subject of the invention is therefore the formula (I) compounds in the form of pure enantiomers or pure diastereoisomers or in the form of a mixture of enantiomers, notably racemates, or mixtures of diastereoisomers.

It follows from the aforesaid that the CONH₂ substituent and the second cycle nitrogen atom may lie in cis and/or trans position relative to the 6-member cycle to which they are attached, and that the subject of the invention therefore concerns formula (I) compounds in the form of cis isomers or trans isomers or mixtures.

The subject of the invention is notably:
trans-(1R,2S,5R)-6-(1-fluoro-2-hydroxy-2-oxoethoxy)-7-oxo-1,6-diazabicyclo[3.2. 1]octane-2-carboxamide,
trans-(1R,2S,5R)-6-(1,1-difluoro-2-hydroxy-2-oxoethoxy)-7-oxo-1,6-diazabicyclo[3.2.1]octane-2-carboxamide,
   in free form, in the form of zwitterions and salts with pharmaceutically acceptable mineral or organic bases.

According to one variant, the compounds:
trans-(1R,2S,5R)-6-(1-fluoro-2-hydroxy-2-oxoethoxy)-7-oxo-1,6-diazabicyclo[3.2.1]octane-2-carboxamide,
trans-(1R,2S,5R)-6-(1,1-difluoro-2-hydroxy-2-oxoethoxy)-7-oxo-1,6-diazabicyclo[3.2.1]octane-2-carboxamide,
   are in the form of their sodium salts.

A further subject of the invention is a method to prepare formula (I) compounds, characterized in that a formula (II) compound:

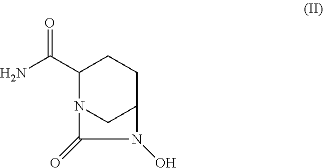

is treated with a reagent of formula (III):

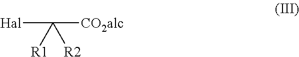

wherein R₁ and R₂ are defined as previously, Hal represents a halogen atom different from fluorine and alc represents an alkyl radical containing 1 to 6 carbon atoms, in the presence of a base, to obtain the compound of formula (IV):

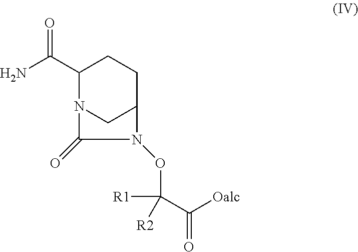

whose ester function is hydrolyzed to obtain the corresponding acid of formula (I) which, if desired, may be salified.

Under preferred conditions to implement the method of the invention, Hal is a bromine atom.

The base used may be an alkaline carbonate or bicarbonate or an amine base, an alkaline carbonate being preferred.

It is possible to operate in a solvent such as dimethylformamide, tetrahydrofuran or acetonitrile.

Hydrolysis of the ester can be conducted using saponification by action of an alkaline hydroxide e.g. lithium or sodium hydroxide in tetrahydrofuran, or of a tetrahydrofuran-water mixture, or by acid hydrolysis using trifluoroacetic acid for example in particular if the ester is a tert-butyl ester.

The compounds of the invention have remarkable β-lactamases inhibiting properties and hence are of interest as medicinal products to combat or prevent infectious diseases, in the form of a combination with various antibiotic compounds of β-lactamines type, to strengthen their efficacy in the fight against pathogenic bacteria producing β-lactamases.

It is well known that the enzyme inactivation of antibiotics of β-lactamines type, whether compounds of penicillin type or cephalosporins, in the treatment of bacterial infections, is an obstacle for these types of compounds. This inactivation consists of a degradation process of the β-lactamines and forms one of the mechanisms for which bacteria can become resistant to treatments. It is therefore desirable to arrive at countering this enzymatic process by associating with the antibacterial agent of β-lactamines type an agent capable of inhibiting the enzyme. When a β-lactamases inhibitor is used in combination with an antibiotic of β-lactamines type, it may therefore reinforce its efficacy against some microorganisms.

The β-lactamases inhibitory activity of the formula (I) compounds is noteworthy and unexpected in particular if it is compared with that of the corresponding non-fluorinated compound. A preparation of this reference compound is described below under Example 1. A table of the compared activities is given later in the application.

A further subject of the invention—as medicinal products and notably medicinal products intended for the treatment of bacterial infections in man or animal via inhibited production of β-lactamases by pathogenic bacteria—therefore concerns the formula (I) compounds such as defined above and their salts with pharmaceutically acceptable acids and bases, and notably the two above-mentioned compounds.

The antibiotic of β-lactamines type with which a formula (I) compound may be combined can be chosen from the group comprising penams, penems, carbapenems, cephems, carbacephem, oxacephems, cephamycin and monobactams.

By β-lactamines is meant penicillins for example, such as amoxicillin, ampicillin, azlocillin, mezlocillin, apalcillin, hexacillin, bacampicillin, carbenicillin, sulbenicillin, ticarcillin, piperacillin, azlocillin, mecillinam, pivmecillinam, methicillin, ciclacillin, talampicillin, aspoxicillin, oxacillin, cloxacillin, dicloxacillin, flucloxacilline, nafcillin or pivampicillin, cephalosporins such as cephalothin, cephaloridine, cefaclor, cefadroxil, cefamandole, cefazolin, cephalexin, cephradine, ceftizoxime, cefoxitin, cephacetril, cefotiam, cefotaxim, cefsulodin, cefoperazone, ceftizoxim, cefmenoxim, cefmetazol, cephaloglycin, cefonicid, cefodizim, cefpirom, ceftazidim, ceftriaxone, cefpiramid, cefbuperazone, cefozopran, cefepim, cefoselis, cefluprenam, cefuzonam, cefpimizol, cefclidin, cefixim, ceftaroline, ceflibutene, cefdinir, cefpodoxime axetil, cefpodoxime proxetil, cefteram pivoxil, cefetamet pivoxil, cefcapene pivoxil or cefditoren pivoxil, cefuroxime, cefuroxime axetil, loracarbacef, latamoxef, carbapenems such as imipenem, meropenem, biapenem or panipenem and monobactams such as aztreonam and carumonam, and their salts. Among the cephalosporins, ceftazidime is particularly preferred.

The formula (I) compounds or their pharmaceutically acceptable salts can be administered at the same time as the antibiotics of β-lactamines type, or separately, preferably after the antibiotic. This can be achieved in the form of a mixture of two active ingredients or in the form of a pharmaceutical association of the two separate active ingredients.

The dosage of the formula (I) compounds and of their pharmaceutically acceptable salts may evidently vary within broad limits and must evidently be adapted, in each particular case, to individual conditions and to the pathogenic agent to be treated. In general, for use in the treatment of bacterial infections, the daily dose may range from 0.250g to 10g per day, via oral route in man, with the product described in Example 3, or from 0.25 g to 10g per day via intramuscular or intravenous route. For use as β-lactamases inhibitor, a daily dose ranging from 0.1 to about 10g may be suitable.

Also, the ratio of the a β-lactamases inhibitor of formula (I), or of the pharmaceutically acceptable salt thereof, to the antibiotic of β-lactamines type may also vary within broad limits and is to be adapted, in each particular case, to individual conditions. In general a ratio ranging from about 1:20 to about 1:1 is indicated.

Medicinal products inhibiting β-lactamases such as defined above are implemented in the form of pharmaceutical compositions in a mixture with an inert pharmaceutical excipient, whether organic or mineral, adapted to the desired administration mode, and a further subject of the invention concerns pharmaceutical compositions containing as active ingredient at least one of the compounds of the invention such as defined above and combinations of the compounds of the invention with β-lactamines.

These compositions can be administered via oral, rectal, parenteral, notably muscular, route or via local route as topical application to the skin and mucosa.

These compositions may be solid or liquid and in pharmaceutical forms commonly used in human medicine, such as ordinary or coated tablets, capsules, granules, suppositories, preparations for injection, ointments, creams, gels; they are prepared following usual methods. The active ingredient(s) can be incorporated therein with excipients usually used in these pharmaceutical compositions such as talc, gum arabic, lactose, starch, magnesium stearate, cocoa butter, aqueous or non-aqueous vehicles, fats of animal or plant origin, paraffin derivatives, glycols, various wetting agents, dispersants or emulsifiers and preserving agents.

These compositions may also be in the form of a lyophilisate intended to be dissolved extemporaneously in a suitable vehicle e.g. sterile apyrogenous water.

The following examples illustrate the invention.

### EXPERIMENTAL PART

### Example 1

### Preparation of trans-(1R,2S,5R)-6-(2-hydroxy-2-oxoethoxy)-7-oxo-1,6-diazabicyclo[3.2.1]octane-2-carboxamide (reference compound).

### Stage A

### trans-(1R,2S,5R)-6-(2-ethoxy-2-oxoethoxy)-7-oxo-1,6-diazabicyclo[3.2.1]octane-2-carboxamide

Ethyl bromoacetate (200 μL, 17.8 mmol) is added to a suspension of K₂CO₃ (270 mg, 19.4 mmol) and trans-(1R,2S,5R)-6-hydroxy-7-oxo-1,6-diazabicyclo[3.2.1]octane-2-carboxamide (racemic described under Stage B of Example 33a in WO 03/063864) (300 mg, 16.2 mmol) in dimethylformamide (0.6 mL) under nitrogen. The reaction medium is agitated 24 h at ambient temperature. The suspension is then diluted with ethyl acetate and filtered. The filtrate (about 60 mL) is washed in water. The combined organic phases are dried then concentrated under reduced pressure. The oil obtained is separated by silica chromatography using as eluent dichloromethane/methanol 99:1 to 95:5, to yield colourless oil (223 mg, 46%).

¹H NMR (400 MHz, MeOH-d₄): δ (ppm)=4.56-4.69 (m, 2H, OCH₂CO₂CH₂C₁₋₁₃), 4.26 (q, 2H, OCH₂CO₂CH₂CH₃), 4.11 (m, 1H, CHCONH₂), 3.94 (m, 1H, NCH₂CHN), 3.13 (m, 1H, NCH₂CHN), 3.03 (m, 1H, NCH₂CHN), 2.29 (m, 1H, CH₂CH₂), 2.14 (m, 1H, CH₂CH₂), 1.93 (m, 1H, CH₂CH₂), 1.81 (m, 1H, CH₂CH₂), 1.33 (t, 3H, OCH₂CO₂CH₂CH₃).

### Stage B

### trans-(1R,2S,5R)-6-(2-hydroxy-2-oxoethoxy)-7-oxo-1,6-diazabicyclo[3.2.1]octane-2-carboxamide

The product obtained at Stage A (129 mg, 4.8 mmol) is dissolved in 8 mL tetrahydrofuran. The solution is diluted with 2.7 mL water then cooled to 0° C. L10H,H₂O (21 mg, 5.0 mmol) is added to the solution. Agitation is continued for 30 min at 0° C. Saponification is halted through the addition of 300 μL of an aqueous 2N HCl solution. The reaction mixture is gradually diluted with ethyl acetate (50 mL) and agitated for 30 min whilst allowing the temperature to rise to 10° C. After decanting the aqueous phase is re-extracted with ethyl acetate. The combined organic phases are dried then concentrated under reduced pressure. The product obtained is dried in vacuo to give an amorphous solid (76 mg, 66%).

MS (ES(+)): m/z [M+H]⁺=244; [2M+H]⁺=287.

¹H NMR (400 MHz, MeOH-d₄): δ (ppm)=4.57-4.66 (m, 2H, OCH₂CO₂H), 4.14 (m, 1H, CHCONH₂), 3.94 (d, 1H, NCH₂CHN), 3.16 (m, 1H, NCH₂CHN), 3.04 (m, 1H, NCH₂CHN), 2.29 (dd, 1H, CH₂CH₂), 2.16 (m, 1H, CH₂CH₂), 1.93 (m, 1H, CH₂CH₂), 1.79 (m, 1H, CH₂CH₂).

### Example 2

### trans-(1R,2S,5R)-6-(1-fluoro-2-hydroxy-2-oxoethoxy)-7-oxo-1,6-diazabicyclo[3.2.1]octane-2-carboxamide

### Stage A

### trans-(1R,2 S,5R)-6-(2-ethoxy-1-fluoro-2-oxoethoxy)-7-oxo-1,6-diazabicyclo[3.2.1]octane-2-carboxamide

Substitution is performed under the same conditions as those described under reference Example 1 using as starting products ethyl bromofluoroacetate (730 μL, 5.94 mmol), K₂CO₃ (896 mg, 6.48 mmol) and the isomer (1R,2S,5R) of the Stage B compound in Example 33a of WO 03/063864 (1 g, 5.40 mmol) in 2 mL dimethylformamide. After treatment, the oil obtained is separated by silica chromatography using as eluent dichloromethane/methanol 98:2 to 95:5 yielding 1.18 g of product with estimated HPLC purity of 71%. The product is a mixture of 2 diastereoisomers in a ratio of 1:1. The product undergoes a second chromatography to yield a colourless oil (975 mg, 62%).

¹H NMR (400 MHz, MeOH-d₄): δ (ppm)=6.02/6.09 (d, 1H, OCHFCO₂CH₂CH₃), 4.29 to 4.37 (m, 2H, OCHFCO₂CH₂CH₃), 3.98 to 4.03 (m, 2H, CHCONH₂ and NCH₂CHN), 3.10 to 3.21 (m, 2H, NCH₂CHN), 2.28 (m, 1H, CH₂CH₂), 2.10 (m, 1H, CH₂CH₂), 1.89 to 1.96 (m, 2H, CH₂CH₂), 1.32 to 1.39 (m, 3H, OCHFCO₂CH₂CH₃).

### Stage B

### trans-(1R,2S,5R)-6-(1-fluoro-2-hydroxy-2-oxoethoxy)-7-oxo-1,6-diazabicyclo[3.2.1]octane-2-carboxamide

Saponification is conducted under the same conditions as those described under Example 1 Stage B using as starting products the ester obtained previously (298 mg, 1.03 mmol), tetrahydrofuran (12 mL), water (4 mL) and LiOH, H₂O (45 mg, 1.08 mmol) Agitation is continued for 1 h at 0° C. The reaction treatment leads to an amorphous yellow product (231 mg, 84%).

MS (ES(−): m/z [M−H]⁻=260; [M+HCOOH−H]⁻=306; [2M−H]⁻=521.

¹H NMR (400 MHz, MeOH-d₄): δ (ppm)=5.96/6.03 (d, 1H, OCHFCO₂H), 3.99 to 4.05 (m, 2H, CHCONH₂ et NCH₂CHN), 3.10 to 3.20 (m, 2H, NCH₂CHN), 2.28 (m, 1H, CH₂CH₂), 2.13 (m, 1H, CH₂CH₂), 1.84 to 2.01 (m, 2H, CH₂CH₂).

### Example 3

### Sodium salt of trans-(1R,2S,5R)-6-(1,1-difluoro-2-hydroxy-2-oxoethoxy)-7-oxo-1,6-diazabicyclo[3.2.1 ]octane-2-carboxamide

### Stage A

### trans-(1R,2S,5R)-6-(1,1-difluoro-2-ethoxy-2-oxoethoxy)-7-oxo-1,6-diazabicyclo[3.2.1]octane-2-carboxamide

Substitution is conducted under the same conditions as those described under Example 1 Stage 1 using as starting products ethyl bromodifluoroacetate (1.4 mL, 10.9 mmol), K₂CO₃ (1.21 g, 8.8 mmol) and the isomer (1R,2S,5R) of the Stage B compound in Example 33a of WO 03/063864 (690 mg, 3.73 mmol) in dimethylformamide (4 mL). The oil obtained after treatment is separated by silica chromatography using as eluent dichloromethane/methanol 99:1 to 95:5 to yield a colourless oil which tends to crystallize (505 mg, 44%). The residual solid is triturated in isopropyl ether to give a white solid (361 mg).

MS (ES(+)): m/z [M+H]⁺=308.1; [M+CH3CN+H]⁺=349; [2M+H]⁺=615.

¹H NMR (400 MHz, MeOH-d₄): δ (ppm)=4.44 (q, 2H, OCF₂CO₂CH₂CH), 3.99 to 4.06 (m, 2H, CHCONH₂ and NCH₂CHN), 3.28 (m, 1H, NCH₂CHN), 3.18 (d system AB, 1H, NCH₂CHN), 2.29 (m, 1H, CH₂CH₂), 2.10 (m, 1H, CH₂CH₂), 1.89 to 2.00 (m, 2H, CH₂CH₂), 1.40 (t, 3H, OCF₂CO₂CH₂CH₃).

### Stage B

### trans-(1R,2S,5R)-6-(1,1-difluoro-2-hydroxy-2-oxoethoxy)-7-oxo-1,6-diazabicyclo[3.2.1]octane-2-carboxamide

Saponification is conducted under the same conditions as those described under Example 1 Stage B using as starting products the ester previously obtained (110 mg, 0.36 mmol), tetrahydrofuran, water (2 mL) and LiOH, H₂O (16 mg, 0.38 mmol). Agitation was continued for 1 h at 0° C. The reaction treatment leads to a white solid (73 mg, 73%).

MS (ES(−)): m/z [M−H]⁻=278; [M+HCOOH−H]⁻=324; [2M−H]⁻=557.

¹H NMR (400 MHz, MeOH-d₄): δ (ppm)=3.99 to 4.05 (m, 2H, CHCONH₂ and NCH₂CHN), 3.29 (m, 1H, NCH₂CHN), 3.17 (m, 1H, NCH₂CHN), 2.29 (m, 1H, CH₂CH₂), 2.12 (m, 1H, CH₂CH₂), 1.95 to 2.04 (m, 2H, CH₂CH₂).

### Stage C

### Triethylamine salt of trans-(1R,2S,5R)-6-(1,1-difluoro-2-hydroxy-2-oxoethoxy)-7-oxo-1,6-diazabicyclo[3.2.1]octane-2-carboxamide

The compound obtained at Stage B is dissolved in 3 mL tetrahydrofuran. Triethylamine (70 μL, 0.5 mmol) is added dropwise to the solution cooled in an ice bath. A precipitate is formed. Agitation is continued for 1 h. The suspension is diluted with THF and filtered to give a white solid (100 mg, 55%)

¹H NMR (400 MHz, MeOH-d₄): δ (ppm)=4.01 to 4.04 (m, 2H, CHCONH₂ and NCH₂CHN), 3.24 to 3.30 (m, 7H, 1 H of NCH₂CHN and 6H of (CH₃CH₂)₃N), 3.14 (d, 1H, NCH₂CHN), 2.30 (m, 1H, CH₂CH₂), 2.22 (m, 1H, CH₂CH₂), 2.03 (m, 1H, CH₂CH₂), 1.86 (m, 1H, CH₂CH₂), 1.37 (t, 9H, (CH₃CH₂)₃N).

### Stage D

### Sodium salt of trans-(1R,2S,5R)-6-(1,1-difluoro-2-hydroxy-2-oxoethoxy)-7-oxo-1,6-diazabicyclo[3.2.1]octane-2-carboxamide

A suspension of 10g DOWEX 50WX8 resin in 2N sodium hydroxide solution (50 mL) is agitated for 1 h, then poured through a chromatography column. The column is packed with demineralized water to neutral pH, then with water/THF 90:10 mixture. The salt obtained at Stage C of Example 3 (50 mg, 0.13 mmol) is dissolved in minimum water/THF solution, placed in the column then eluted with a water/THF 90:10 mixture. The fractions containing the substrate are combined and frozen. The frozen solution is lyophilized to arrive at the expected sodium salt (39 mg, 97%) in the form of a white solid.

MS (ES(−)): m/z [M−H]⁻=278; [M+HCOOH−H]⁻=324; [2M−H]⁻=557.

¹H NMR (400 MHz, MeOH-d₄): δ (ppm)=4.01 (m, 2H, CHCONH₂ and NCH₂CHN), 3.29 (m, 1H, NCH₂CHN), 3.11 (d, 1H, NCH₂CHN), 2.27 (m, 1H, CH₂CH₂), 2.17 (m, 1H, CH₂CH₂), 2.00 (m, 1H, CH₂CH₂), 1.84 (m, 1H, CH₂CH₂).

### Study of the β-Lactamase Inhibiting Activity of the Compounds of the Invention

I/ The formula (I) compounds and their pharmaceutically acceptable salts have marked inhibitory activity against the β-lactamases of various bacterial strains, and these properties of therapeutic interest can be determined in vitro on isolated β-lactamases:

A. Preparation of Tem-1 and P99 β-lactamases

The β-lactamases are isolated from bacterial strains resistant to penicillins and cephalosporins (Tem 1 and P99 are respectively produced by E. coli 250HT21 and E.Cloacae 293 HT6).

The bacteria are cultured in 37g/l brain-heart infusion (DIFCO), at 37° C. They are harvested at the end of the exponential phase, cooled and centrifuged. The bacterial residues are dissolved in 50 mM sodium phosphate buffer pH 7.0 and again centrifuged. The bacteria are dissolved in two volumes of this same buffer and lysed using a French-Press held at 4° C. After centrifuging 1 h at 100 000g, at 4° C., the supernatants containing the soluble fraction of the bacterial extracts are collected and frozen to −80° C.

B. Determination β-lactamase Activity

The method uses Nitrocefin (OXOID) as substrate, a chromogenic cephalosporin, whose hydrolysis product by β-lactamases is red and absorbed at 485 nm.

β-lactamase activity is determined kinetically by measuring the variation in absorbance at 485 nm resulting from hydrolysis of the substrate on a plate spectrophotometer (Spectra Max Plus by Molecular Devices). Experiments are conducted at 37° C. The quantity of enzyme was normalized and measurements are taken at initial velocity.

C. Determination of β-Lactamase Inhibiting Activity

The measurements are taken with pre-incubation of the enzyme and of the inhibitor (5 min). The products are assayed at 11 concentrations. The reaction mixture contains 100 μM Nitrocefin and 50 mM sodium phosphate buffer, pH 7.0, and 0.1 mg/mL bovine serum albumin.

D. IC50 Calculations

Hydrolysis velocities are measured with and without inhibitor. The inhibitor concentration is determined which inhibits 50% of the Nitrocefin hydrolysis reaction by the enzyme (IC50). Data is processed using GraFit software (Erithacus Software). The IC50 values are the mean of the IC50s obtained in at least 2 different experiments.

IC50 (μM) enzyme inhibition

| β-lactamase | Example 1 | Example 2 | Example 3 |
|---|---|---|---|
| E. coli TEM1 | 0.72 | 0.002 | 0.0008 |
| E. cloacae P99 | 54 | 4.6 | 2.2 |

II/ The evidenced β-lactamase inhibiting activity potentialises the antibacterial activity of antibiotics of β-lactam type, and therefore leads to a synergic effect, as is shown by the results given below which express the minimum inhibitory concentration in vitro (MIC in μg/ml), against a certain number of pathogenic microorganisms, and of combinations of ceftazidime (<<CAZ >>) with formula (I) compounds at a concentration of 4 mg/l. Operations are as follows using the so-called micro-dilution method in liquid medium.

A series of β-lactam concentrations are prepared in the presence of a constant concentration (4 mg/l) of the product to be assayed, each is then seeded with various bacterial strains.

After 24-hour incubation in an oven at 37° C. growth inhibition is assessed via the lack of any bacterial growth, which allows determination of the minimum inhibitory concentrations (MICs) for each strain, expressed in milligrams/l.

The results obtained are given in the following Table:

Restoration of MICs in combination with CAZ (μg/mL)
24 h (inhibitory concentration 4 μg/mL)

| | | | | | CAZ-Inhibitor | | |
|---|---|---|---|---|---|---|---|
| Assay | Strain | | Phenotype | CAZ alone | Example 1 | Example 2 | Example 3 |
| 1 | 250BE6 | E. coli | TEM3 | >32 | 2 | 0.25 | 0.25 |
| 2 | 293HT6 | E. cloacae | P99 AmpC | >32 | >32 | 8 | 1 |
| 3 | 293HT4 | E. cloacae | AmpC | >32 | >32 | 8 | 0.5 |
| 4 | 261GR6 | C. freundii | AmpC | >32 | >32 | 4 | 1 |

### Examples of Pharmaceutical Compositions

1) A pharmaceutical composition for injection was
prepared having the following ingredients:

| | | |
|---|---|---|
| Compound of Example 2 | 500 | mg |
| Sterile aqueous excipient | QS to 10 | ml |

2) A pharmaceutical composition for injection
(lyophilisate) was prepared containing:

| | | |
|---|---|---|
| Compound of Example 3 | 500 | mg |
| Ceftazidime | 1 | g |
| sterile aqueous excipient | QS to 5 | ml |

The two active ingredients can, if desired, be added separately in two different ampoules or bottles.

1) The compounds meeting formula (I): 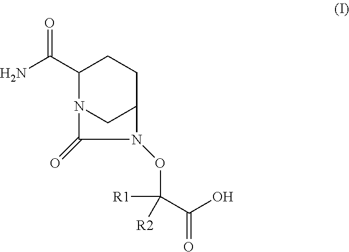
wherein one of R₁ and R₂ is a hydrogen and the other a fluorine or both represent a fluorine, in free form, in the form of zwitterions and in salt form with pharmaceutically acceptable mineral or organic bases.

2. The compounds according to claim 1 having the following names:
trans-(1R,2S,5R)-6-(1-fluoro-2-hydroxy-2-oxoethoxy)-7-oxo-1,6-diazabicyclo[3.2.1]octane-2-carboxamide,
trans-(1R,2S,5R)-6-(1,1-difluoro-2-hydroxy-2-oxoethoxy)-7-oxo-1,6-diazabicyclo[3.2.1]octane-2-carboxamide,
in free form, in the form of zwitterions and in salt form with pharmaceutically acceptable mineral or organic bases.

3) The compounds according to claim 2 in the form of their sodium salts.

4) Method to prepare formula (I) compounds characterized in that a formula (II) compound: 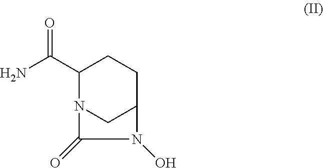
is treated with a reagent of formula (III): 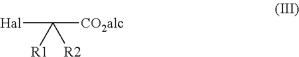
wherein R₁ and R₂ are defined as in claim 1, Hal represents a halogen atom different from fluorine and alc represents an alkyl radical containing 1 to 6 carbon atoms, in the presence of a base, to obtain the compound of formula (IV): 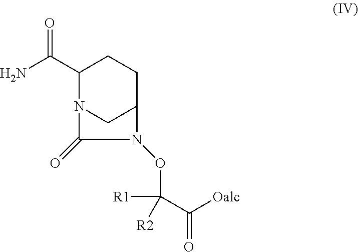
whose ester function is hydrolyzed to obtain the corresponding acid of formula (I) which, if desired, is salified.

5) The formula (I) compounds such as defined in claim 1 as medicinal products.

6) The compounds of claim 2 as medicinal products.

7) Pharmaceutical compositions, characterized in that as active ingredient they contain at least one medicinal product according to claim 5.

8) Pharmaceutical compositions according to claim 7, further containing at least one medicinal product of β-lactamines type.

9) Combinations of a β-lactamases inhibitor medicinal product according to claim 5 with a medicinal product of β-lactamines type.

10) Combinations according to claim 9 wherein the medicinal product of β-lactamines type is a cephalosporin or a carbapenem.

11) Combinations according to claim 10 wherein the cephalosporin is ceftazidime.
